(19) United States
(12) Patent Application Publication
(10) Pub. No.: US 2004/0152666 A1
(43) Pub. Date: Aug. 5, 2004

(54) Methods of reducing complications associated with peritoneal dialysis in patients with diabetes obesity and/or hyperlipidemia

(76) Inventors: Paul Tam, North York (CA); George Wu, North York (CA)

Correspondence Address: GREENLEE WINNER AND SULLIVAN P C, 5370 MANHATTAN CIRCLE, SUITE 201, BOULDER, CO, 80303, US

(21) Appl. No.: 10/732,009

(22) Filed: Dec. 9, 2003

Related U.S. Application Data
(60) Provisional application No. 60/432,466, filed on Dec. 10, 2002.

(51) Int. Cl. A61K 31/7008

(52) U.S. Cl. 514/62

(57) ABSTRACT

The present invention relates to a method of treating a patient suffering from renal failure in combination with diabetes, obesity, hyperlipidemia, or a combination thereof, by empolying a peritoneal dialysis solution comprising N-acetylglucosamine as an osmotic agent. The use of N-acetylglucosamine as an osmotic agent, singly or in combination with other osmotic agent(s), in the dialysis fluid minimizes the risk of or prevents hyperglycemia, hyperinsulinemia, hypertension, and other complications associated with the current practice of peritoneal dialysis in such patients.

### CROSS-REFERENCE TO RELATED APPLICATION

This application claims the benefit of U.S. Provisional Application No. 60/432,466, filed Dec. 10, 2002, which is hereby incorporated by reference to the extent not inconsistent with the disclosure herewith.

### BACKGROUND OF THE INVENTION

The present invention relates to a method of treating a patient suffering from renal failure by performing peritoneal dialysis, in particular, patients with diabetes, obesity and/or hyperlipidemia.

Diabetes is the most common cause of end stage renal failure. Peritoneal dialysis is an alternative to hemodialysis for renal replacement therapy in that group of patients. It does not require vascular access which is often problematic in diabetic patients with advanced atherosclerosis. If effective, the treatment of peritoneal dialysis should serve to restore and maintain the composition of the blood in such patients within a reasonable range. The main drawback of peritoneal dialysis in diabetics is that glucose is used as osmotic solute in most of the contemporarily available dialysis fluids, and this creates additional metabolic problems in diabetic patients. That is why there is a continuous search for an alternative to glucose osmotic solute which could be safely used in diabetic peritoneal dialysis patients.

Various osmotic solutes such as amino acids, xylitol, gelatin, glycerol, polypeptides or polyglucose have been proposed as replacement for glucose in peritoneal dialysis (1). However, none of them is ideal and without potential side effects.

Glycerol, which was proposed in Europe as an osmotic solute for diabetic patients, shows advantages over glucose: low insulin requirement (2) and improved biocompatibility (3), however, it also has several disadvantages: low transperitoneal ultrafiltration (4) and danger of blood hyperosmolality (5).

Polyglucose is much more effective than glycerol in inducing transperitoneal ultrafiltration (6). However, its metabolism results in accumulation of maltose in the body (7) and recently several reports describing severe dermatitis after use of icodextrin were published (8, 9).

There is a continuing need to identify a suitable osmotic agent for the preparation of the peritoneal dialysis fluid useful for preforming peritoneal dialysis, in patients suffering renal failure in combination with diabetes, obesity, and/or hyperlipidemia, by which the required osmotic pressure can be achieved without the secondary problems referred to above. An appropriate osmotic agent should have the following properties: it should satisfy the needs for peritoneal dialysis, i.e., be a non-toxic substance and the accumulation of unacceptable derivatives or metabolites in the peritoneum or in the circulation should be avoided; it should not rapidly cross the peritoneal membrane into the blood and in this respect it should allow maintenance of the required untrafiltration; it should not react with the peritonium or with proteins, leading to secondary reactions involving peritoneal membrane, of peritoneal cells, or of cells in the circulation; it should not alter cell function which can reduce natural local phagocytosis, and the ability of the immune system to kill bacteria. The inventors herein discovered such an osmotic agent, N-acetylglucosamine (NAG), which was described previously (U.S. Pat. No. 6,083,935). The present invention concerns the use of NAG in the peritoneal dialysis procedure in a patient, particularly one with diabetes, obesity and/or hyperlipidemia such that complications associated with such diseases, especially those related to hyperglycemia, hyperinsulinemia, and/or hypertension can be prevented or minimized.

### SUMMARY OF THE INVENTION

The present invention provides a method of treating a patient suffering from renal failure in combination with diabetes, obesity, and/or hyperlipidemia by performing peritoneal dialysis using the dialysis fluid comprising N-acetylglucosamine (NAG). The peritoneal dialysis fluid useful for the invention has a pH typically in the range of about 5.0 to 8.0 and contains an osmotically effective amount of NAG at a concentration of between about 0.5 to 5.0% (w/v) and physiologically acceptable electrolytes at appropriate concentrations as are known in the art. Optionally, such peritoneal dialysis fluid can contain at least one additional osmotic agent that is known in the art. Examples include but are not limited to glucose, iduronic acid, glucuronic acid, and combinations thereof. As demonstrated herein, the peritoneal dialysis fluids comprising NAG show superior properties, i.e., resulting in lower blood glucose and insulin levels, compared to the currently available peritoneal dialysis fluids when used in the aforementioned conditions. Thus, the invention is useful in preventing or reducing complications associated with diabetes, obesity and/or hyperlipidemia, especially those related to hyperglycemia, hyperinsulinemia, and/or hypertension.

### BRIEF DESCRIPTION OF THE DRAWINGS

FIG. 1 shows the dialysate volume (A) and dialysate/serum×1000 (D/S×1000) ratio for total protein (B) during acute peritoneal dialysis in rats performed with glucose-based (white bars) or NAG-based (black bars) dialysis fluids with dwell time of 1, 3 and 6 hours.

FIG. 2 shows the changes in blood glucose (A) and insulin (B) concentrations during acute peritoneal dialysis in rats performed with glucose-based (white bars) or NAG-based (black bars) dialysis fluids with dwell time of 1,3 and 6 hours.

### DETAILED DECSRIPTION OF THE INVENTION

In general the terms and phrases used herein have their art-recognized meaning, which can be found by reference to standard texts, journal references and contexts known to those skilled in the art.

The terms, “peritoneal dialysis fluid” and “peritoneal dialysis solution”, are used interchangeably in the present application and intended to indicate a physiologically acceptable aqeous solution which contains physiologically acceptable inorganic anions and cations, and at least one osmotic agent at concentrations sufficient for efficient removal of water and solutes from a patient by peritoneal dialysis. Examples of the anions and cations include, but are not limited to, Na⁺, K⁺, Ca⁺⁺, Cl⁻ and lactate ions. Typically, the peritoneal dialysis solution of the invention has a pH of 5.0-8.0, sodium at a concentration of 115-140 mEquiv/L, calcium at a concentration of 0.6-3.24 mEquiv/L, chloride at a concentration of 100-145 mEquiv/L, magnesium at a concentration up to 2.0 mEquiv/L and lactate, malate, acetate or succinate at a concentration of 30-45 mEquiv/L in addition to the osmotic agent present therein.

The present invention provides a method for treating patients with renal failure in combination with diabetes, obesity, and hyperlipidemia using a peritoneal dialysis (PD) fluid comprising N-acetylglucoseamine as an osmotic agent, singly or in combination with other osmotic agents (e.g., glucose). The patients are preferably humans but also include other mammals who are in need of a peritoneal dialysis treatment. One of ordinary skill in the art will understand that the PD fluids of the invention further comprise other physiologically acceptable inorganic cations and anions in the appropriate range of concentrations and pH, and pharmacuetically acceptable additives, that are well known in the art. The terms such as “dialysis fluid” and “dialysis solution”, as well as “osmotic agent” and “osmotic solute” are used interchangeably herein.

In order to identify a better osmotic agent which can be used with minimum adverse effects in the peritoneal dialysis procedure in patients with diabetes, obesity, and hyperlipidemia, the inventors herein evaluated N-acetylglucosamine (NAG) and compared it to another currently used osmotic agent, glucose, during peritoneal dialysis in rats.

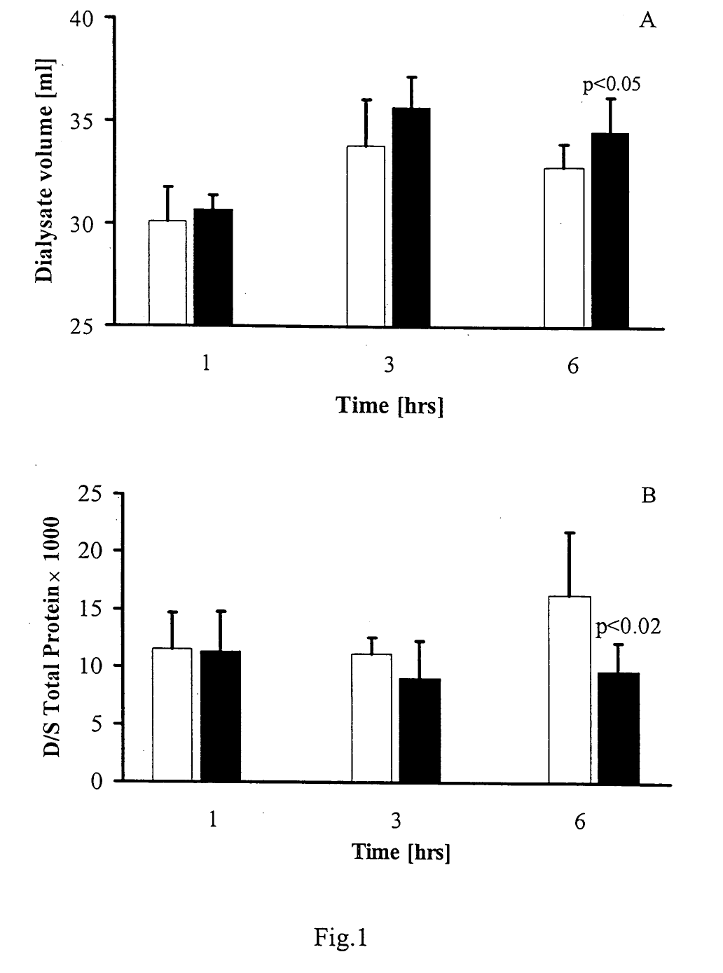

Studies illustrated in detail below demonstrate that during 6 hour exchange end dialysate volume was higher in a group of rats dialysed with NAG (220 mmol/l) containing fluid than that in those dialysed with fluid containing glucose (220 mmol/l) (GLU): 34.5±1.7 ml vs. 32.8±1.1 ml, p<0.05. Also peritoneal permeability to protein (D/S×1000) was lower in NAG group: 9.7±2.5 vs. 16.3±5.6 in GLU, p<0.02. Dialysis with GLU-based dialysis fluid resulted in blood glucose concentration up to 180±39 mg/dl whereas in the NAG group the increase of blood glucose level was much lower, the highest observed being 91±9 mg/dl; p<0.001, as shown in FIG. 1A. Dialysis with GLU-fluid caused an increase of blood insulin level by 53.2±62.4 pmol/l whereas blood insulin concentration in the NAG group was maximally increased only by 5.0±5.4 pmol/l.

These results indicate that NAG is more effective than glucose as an osmotic agent with reduced peritoneal permeability to protein. Furthermore, the results of lower blood glucose and insulin levels seen in the NAG group are particularly important in diabetic peritoneal dialysis patients since one needs to avoid glucose overloading in these patient. Accordingly, N-acetylglucosamine represents an alternative osmotic solute for use in diabetic patients who are in need of a treatment with peritoneal dialysis. Our studies indicate that N-acetylglucosamine has some beneficial properties of glycerol (low insulin requirement) but at the same time is more effective than glucose or glycerol as an osmotic solute for use in the peritoneal dialysis solution.

The present invention is particularly useful as a method for treating a patient suffering from renal failure in combination with diabetes, obesity and/or hyperlipidemia, wherein said method comprises the introduction of a peritoneal dialysis solution comprising, as osmotic agent, N-acetylglucosamine into the peritoneal cavity of the patient. The use of NAG in the peritoneal dialysis fluids in such instances can prevent or minimize various complications associated with using a conventional peritoneal dialysis solution, in particular, hyperglycemia, hyperinsulinemia and/or hypertension. The terms such as “hyperglycemia”, “hyperinsulinemia” or “hypertension” are well known in the art. Clinical definitions of these terms are provided in standard medical textbooks [e.g., see “Harrison's Principles of Internal Medicine” by Braunwald et al. (2001) McGraw-Hill Professional].

Disclosed herein is a method of reducing complications associated with peritoneal dialysis in combination with diabetes, obesity and/or hyperlipidemia by employing a dialysis fluid comprising NAG. “Reducing” complications means that a particular parameter(s) listed below is improved as measured by the art-recognized means after the peritoneal dialysis using the PD fluid of the invention as compared to that prior to the peritoneal dialysis treatment. The complications that can be prevented or reduced include but are not limited to;

(a) morphological and functional deterioration of the peritoneal membrane;

(b) peritonitis;

(c) adverse metabolic consequences and realted cardiovascular disease;

(d) protein malnutrition;

(e) hyperglycemia;

(f) hyperinsulinemia;

(g) hypertension; and combinations of the any of the above,

most especially hyperglycemia and/or hyperinsulinemia and/or hypertension.

The present inventors found that peritoneal transport was different in rats exposed to NAG-based dialysis fluid (NAG) as compared to animals treated with glucose-based solution (GLU). Dialysate volume were higher during 1 and 3 hrs exchange with NAG based solution and that difference became statistically significant after 6 hours dwell: 34.5±1.7 ml vs. 32.8±1.1 ml in GLU, p<0.05. (FIG. 1A). Peritoneal permeability to total protein expressed as dialysate/serum ratio×1000, declined proportionally to the dwell time in NAG group and after 6 hrs it was lower than that seen in the GLU group: 9.7±2.5 vs. 16.3±5.6 in GLU, p<0.02. (FIG. 1B). Both of the described changes in the peritoneal permeability to water and total protein could be attributed to increased intraperitoneal hyaluronan production in NAG treated rats. In the dialysate obtained after 6 hrs dwell hyaluronan concentration (ng/ml) in the NAG group was 60.3±7.8 vs. 31.4±9.6 in the GLU group, p<0.05. At the same time intraperitoneal inflammation as reflected by the dialysate cell count was comparable in both groups: 870±226 cell/μl in the GLU group and 945±251 cells/μl in the NAG group.

Peritoneal permeability to small solutes was measured only during 1 hour exchange to exclude the effect of bidirectional water flow (ultrafiltration/reabsorption) on transport of solutes. Sodium sieving (D/D₀) was comparable between the two groups: 0.955±0.034 in the NAG group and 0.960±0.029 in the GLU group. Also peritoneal permeability coefficient K (cm×min⁻10⁻⁶) for creatinine was similar in both groups: 7.4±1.4 in the NAG group and 7.8±1.9 in the GLU group.

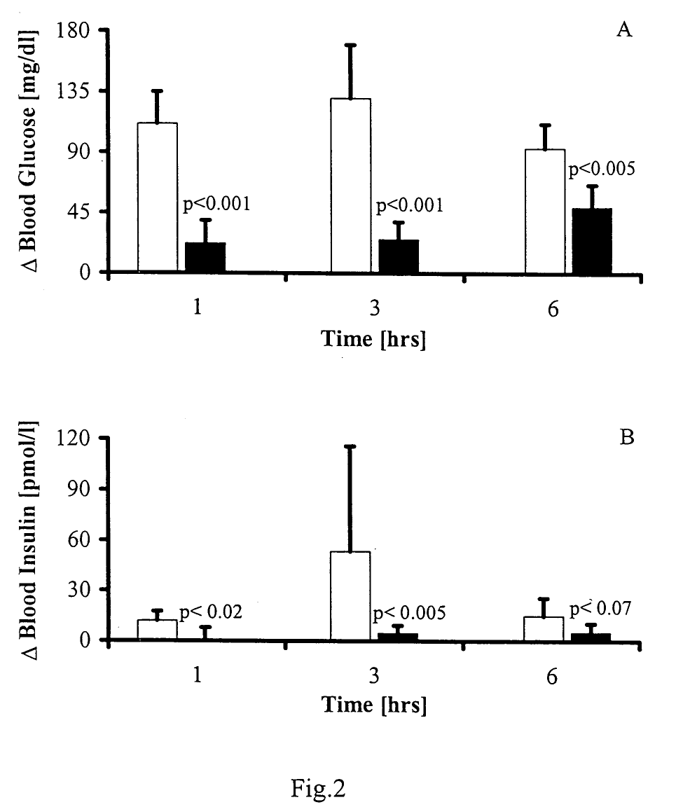

At the beginning of the experiment, blood glucose and insulin concentrations were comparable in all tested groups of rats. Mean value of blood glucose concentration in all rats at that time was equal 48±13 mg/dl and blood insulin 24.2±5.9 pmol/l. During the dwell of the dialysis fluid containing either glucose or NAG blood glucose concentration was increased, however that change was much bigger in rats dialysed with glucose-based dialysis solution. (FIG. 2A). Also blood insulin concentration was increased in all rats during exchange performed with the studied fluids. Increase of blood insulin level was much higher in rats dialysed with the solution containing glucose as an osmotic solute (FIG. 2B).

In summary, the inventors of the present invention have demonstrated that NAG is more effective than glucose as an osmotic solute during peritoneal dialysis in rats as reflected by increased dialysate volume after 6 hours of the intraperitoneal dwell. NAG has slightly larger molecular weight (221 daltons) than that of glucose (180 daltons) and it is likely that part of the difference in net ultrafiltration between the two fluids studied was due to higher reflection coefficient of NAG (6). Significant difference between the dialysate volumes of glucose or NAG-based dialysis fluids observed only after 6 hours dwell indicates that slower reabsorption of fluid from the peritoneal cavity in presence of NAG is responsible for the increased ultrafiltration during NAG dialysis. Increased intraperitoneal synthesis of hyaluronan can explain a decrease of the peritoneal hydraulic permeability which in turn would result in slower fluid reabsorption from the peritoneal cavity. The present inventors have found previously that human peritoneal mesothelial cells increase within six hours synthesis of hyaluronan when exposed in vitro to culture medium supplemented with NAG (12). Increased negative charge of the peritoneum due to accumulation of hyaluronan may also explain, per analogy with glomerulus, reduced transperitoneal loss of protein observed in NAG group (FIG. 1B). In other in vivo experiments in rats the present inventors demonstrated that increased hyaluronan content in the peritoneum results in slower dialysate absorption from the peritoneal cavity and lower transperitoneal loss of protein (13). Rosengren et al. confirmed that hyaluronan reduces back-filtration of fluid from the peritoneal cavity to plasma and transperitoneal transport of albumin (14).

Hyperinsulinemia induced by constant absorption of glucose from the peritoneal dialysis fluid is a characteristic feature in CAPD (Continuous Ammbulatory Peritoneal Dialysis) patients and it is an independent risk factor for ischemic heart disease (15). Further, insulin increases catecholamine secretion and could exacerbate hypertension. Moreover, insulin is known to reduce appetite, which is detrimental in peritoneal dialysis patients where protein malnutrition and poor appetite are commonly seen as side effects. Therefore reduction in the daily glucose load by introducing a new osmotic agent such as NAG which does not stimulate insulin production significantly is particularly beneficial for peritoneal dialysis patients with diabetes, obesity and/or hyperlipidemia. In the present study the inventors observed a small increase in blood insulin level in rats exposed to NAG-based dialysis fluid however that effect was about 12 times weaker than that observed with the dialysis fluid containing glucose, i.e., after 3 hours dialysis blood insulin level was increased by 53.2±62.6 pmol/l in glucose treated rats as compared to 4.5±4.7 pmol/l in animals dialysed with NAG (FIG. 2B). It is possible that the weak stimulation of insulin secretion could be caused by the fact that most of NAG was absorbed directly from the peritoneal cavity into the portal circulation. Because of this insignificant stimulation of insulin secretion, the use of NAG containing dialysis fluid can prevent progress of the atherosclerotic changes as compared to dialysis with glucose-based dialysis solution.

An ideal osmotic solute (agent) used in peritoneal dialysis fluid should be easily metabolized in the body. It was shown previously that intraperitoneal infusion of the radiolabeled NAG is followed by appearance of the tracer in the exhaled air (about 25%) and in urine (about 20%) (19). Significant amount of NAG may also be incorporated into glycoproteins. As shown in the present disclosure, the inventors found that after intraperitoneal infusion of the dialysis fluid containing NAG, blood glucose level was increased proportionally to the dwell time: by 39% after 1 hour, by 54% after 3 hours and by 116% after 6 hours. These findings indicate that NAG absorbed into the portal circulation may be partially metabolized also to glucose. However, several authors reported that intravenous infusion of NAG resulted in none or only slight increase of blood glucose level ( 20) or even in hypoglycemia (16).

In summary, the studies described above indicate that NAG is an excellent osmotic agent for the peritoneal dialysis fluid useful for performing peritoneal dialysis in patients in need of such treatment, particularly those with diabetes, obesity and/or hyperlipidemia. The dialysis fluids containing NAG causes higher net ultrafiltration during peritoneal dialysis as illustrated in the studies in rats due to slower absorption of dialysate from the peritoneal cavity and reduces peritoneal permeability to protein. Additionally NAG given intraperitoneally does not stimulate insulin production to the extent seen with glucose as an osmotic agent, which is critical in uremic diabetic patients treated with peritoneal dialysis. Thus, NAG offeres an alternative to glucose as an osmotic solute in peritoneal dialysis fluid useful for treating a patient suffering from renal failure in combination with diabetes, obesity and hyperlipidemia.

### EXAMPLES

The following examples are provided for illustrative purposes, and are not intended to limit the scope of the invention as claimed herein. The studies described herein are the results observed in an animal model (i.e., rats). However, a person of ordinary skill in the art would understand that certain modifications and adjustments of the invention can readily be made for application in humans and other animals based on the present disclosure. It is understood in the art that the results of the animal studies such as those disclosed herein provide predictive information useful for practicing the invention in human subjects.

Materials and Methods

Peritoneal transport kinetics as well as blood insulin and glucose levels were evaluated in rats during acute peritoneal dialysis. Peritoneal dialysis fluids tested in the studies described herein were prepared in the laboratory and sterilized by filtration. Electrolyte and buffer compositions of both solutions were identical, similar to those contained in commercially available fluids. Concentrations of the individual solutes were as follows (mmol/l): Na 132, Ca 1.75, Mg 0.75, Cl 102, lactate 35. Glucose or NAG were used as osmotic solutes, both in a concentration of 220 mmol/l. Osmolality of the glucose-based dialysis fluid was 478 mOsm/kg H₂0 and of the solution containing NAG 476 mOsm/kg H₂O; pH of the studied fluids was 7.02 and 7.03, respectively.

Acute peritoneal dialysis was performed in male Wistar rats (b.w.270-330 g) according to the methods described previously (see ref. 10 below). For 24 hours prior to the experiments, animals were given only drinking water and no food. At the beginning of the study, under ether anesthesia, blood samples were collected from the tail vein and thereafter 20 ml of the dialysis fluid was infused via a polyvinyl catheter (1.2 gauge; Vygon, Helsingborg, Sweden); the catheter was instantly removed after infusion. Thereafter the rats were transferred to individual cages, signs of anesthesia disappeared within 5 minutes. Dialysis fluid dwelled in the peritoneal cavity for 1 hour, 3 hours or 6 hours. 12 animals were studied at each time interval: 6 in the NAG group and 6 in the glucose group. During the dialysis animals were conscious with free access to drinking water but no food. At the end of the dwell time rats were again anesthetized with ether, blood samples were collected from the heart and then animals were sacrificed by exsanguination from heart. Afterwards the peritoneal cavity was opened and the dialysate was collected with a pipette. Dialysate volume was measured by weight. Blood and dialysate samples were centrifuged (1500 rpm for 10 minutes) and serum or dialysate supernatant were stored at −20° C. for further analysis. In dialysate samples drained after 6 hrs dwell cell count was determined in a Neubauer chamber.

Serum and dialysate samples were analyzed for creatinine (enzymatic test, Analco, Warsaw) and total protein were analyzed using Lowry method (11). Sodium concentration in the unused dialysis fluid and dialysate was measured with flame photometry (Eppendorf, Germany). Glucose concentration in serum was measured with an enzymatic test (Sigma Chemical, St.Louis, Mo., U.S.A.). Insulin concentration in serum was measured with ELISA kit (Mercodia, Uppsala, Sweden). Hyaluronan concentration in the dialysate samples was measured with ELISA (Chugai, Japan). Sodium sieving and permeability coefficient for creatinine were calculated for exchanges with 1 hour dwell. Results are presented as mean±SD. Statistical analysis was performed with Mann-Whitney test for unpaired data. A p value less than 0.05 was considered significant.

### EXAMPLE

In the table below are examples of solutions for use in the method of threatment according to the invention. In solution D-G the osmotic agent NAG has been complemented with glucose. In solution G the buffer part of lactate has been complemented with bicabonate.

| | A | B | C | D | E | F | G |
|---|---|---|---|---|---|---|---|
| Volume (ml) | 2000 | 2080 | 2180 | 1960 | 2000 | 2060 | 2000 |
| Sodium (mM) | 0-140 | 0-140 | 0-140 | 0-140 | 0-140 | 0-140 | 0-140 |
| NAG g/l | 15 | 26 | 38.5 | 5.1 | 8.25 | 12.8 | 5 |
| Lactate (mM) | 38 | 36.5 | 34.9 | 38.8 | 38.0 | 37 | 2.5 |
| Magnesium | 0.24-0.71 | 0.22-0.68 | 0.21-0.65 | 0.24-0.73 | 0.24-0.71 | 0.23-0.69 | 0.24-0.73 |
| (mM) | | | | | | | |
| Calcium (mM) | 0.85-1.9 | 0.82-1.8 | 0.78-1.7 | 0.87-1.9 | 0.86-1.9 | 0.83-1.8 | 0.9-2.0 |
| Glucose g/l | | | | | 16.5 | 25.6 | 10 |
| Bicarbonate | | | | | | | 37.5 |
| mM | | | | | | | |

Those skilled in the art will appreciate that the invention described herein is susceptible to variations and modifications other than those specifically described. It is to be understood that the invention includes all such variations and modifications. For example, a person of ordinary skill in the art would understand that the concentration of NAG can vary when used singly or in combination with another osmotic agent in a given peritoneal dialysis fluid. The invention also includes all of the steps, features, compositions and compounds referred to or indicated in this specification, individually or collectively, and any and all combinations of any two or more of said steps or features. All references cited herein are incorporated by reference to the extent not inconsistent with the disclosure herewith.

References:

1. Khanna R, Peritoneal dialysis in diabetic end-stage renal disease. In: R. Gokal, K D, Nolph eds. The textbook of peritoneal dialysis. Dordrecht, Boston, London, Kluwer Academic Publishers; 1994; 639-659.

2. Heaton A, Ward M K, Johnston D G, Nicholson D V, Alberti K G, Kerr D N. Short-term studies on the use of glycerol as an osmotic agent in continuous ambulatory peritoneal dialysis. Clin. Sci. 1984: 67: 121-130.

3. Breborowicz A, Rodela H, Oreopoulos D G, Toxicity of osmotic solutes on human mesothelial cells in vitro Kidney Int. 1992; 41:1280-1285.

4. Lameire N, Faict D, Peritoneal dialysis solutions containing glycerol and amino acids. Perit. Dial. Int. 1994:14: suppl.3: s145-s151.

5. Matthys E, Dolkart R, Lameire N, Potential hazards of glycerol dialysate in diabetic CAPD patients. Perit.Dial.Bull. 1987:7:16-19.

6. Rippe B, Zakaria E R, Carlsson O, Theoretical analysis of osmotic agents in peritoneal dialysis. What size is and ideal osmotic agent? Perit.Dial.lnt. 1996: 16: suppl.1 s97-s103.

7. Mistry C D, Mallick N P, Gokal R, Ultrafiltration with isosmotic solution during long peritoneal exchanges. Lancet 1987:ii: 178-182.

8. Queffeulou G, Bernard M, Vrtovsnik F, Skhiri H, Lebrun-Vigne B, Severe cutaneous hypersensitivity requiring permanent icodextrin withdrawal in a CAPD patient. Clin. Nephrol. 1999: 51:184-186.

9. Goldsmith D, Jayawardene S, Sabharwal N, Cooney K, Allergic reactions to the polymeric glucose-based peritoneal dialysis fluid icodextrin in patients with renal failure. Lancet 2000: 355:897.

10. Br [symbol] borowicz A, Polubinska A, Moberly J, Ogle K, Martis L, Oreopoulos D G, Hyaluronan modifies inflammatory response and peritoneal permeability during peritonitis in rats. Am. J. Kidney Dis. 2001: 37: 594-600.

11. Lowry O H, Rosenbrough N J, Farr A L, Randall R J, Protein measurement with the folin phenol reagent. J. Biol. Chem. 1951;193:265-275.

12. Br [symbol] borowicz A, Wieczorowska-Tobis K, Kuzlan M et al. N-Acetylglucosamine: a new osmotic solute in peritoneal dialysis solutions. Perit. Dial. Int. 1997 17: suppl.2: s80-s83.

13. Polubinska A, Pawlaczyk K, Ku\dot{z}lan-Pawlaczyk M et al. Dialysis solution containing hyaluronan: effect on peritoneal permeability and inflammation in rats. Kidney Int. 2000: 57: 1182-1189.

14. Rosengren B I, Carlsson O, Rippe B, Hyaluronan and peritoneal ultrafiltration: a test for the “filter-cake” hypothesis. Am. J. Kidney Dis. 2001: 37: 1277-1285.

15. Prichard S, Major and minor risk factors for for cardiovascular disease in peritoneal dialysis. Perit.Dial.lnt. 2000 20 suppl.2:s154-s159.

16. Ashcroft S J H, Ceossley J R, The effects of glucose, N-acetylglucosamine, glyceraldehydes and other sugars on insulin release in vivo. Diabetologia 1975 11:279-284.

17. Zawalich W S, Dye E S, Matschinsky F M, Metabolism and insulin releasing capabilities of glucosamine and N-acetylglucosamine in isolated rat islets. Biochem. J. 1979 180:145-152.

18. Lenzen S, Insulin secretion by isolated perfused rat and mouse pancreas. Am. J. Physiol. 1979: 236: E391-E400.

19. Kohn P, Winzler R J, Hoffman R C, Metabolism of D-Glucosamine and N-Acetyl-D-glucosamine in the intact rat. J.Biol.Chem. 1962: 237: 304-308.

20. Gaulden E C, Keating W C, The effect of intravenous N-Acetyl-D-Glucosamine on the blood and urine sugar concentrations of normal subjects. Metabolism 1964(13):466-472.

We claim:

1. A method of treating a patient suffering from renal failure in combination with diabetes, obesity, hyperlipidemia or a combination thereof, wherein said method comprises introducing a peritoneal dialysis solution comprising N-acetylglucosamine (NAG) as an osmotic agent into the peritoneal cavity of the patient.

2. The method of claim 1 wherein the NAG is present at a concentration of about 0.5 to 5.0% (w/v).

3. The method of claim 2 wherein the peritoneal dialysis solution further comprises an additional osmotic agent selected from the group consisting of glucose, iduronic acid, glucuronic acid, and combinations therof.

4. A method of effecting peritoneal dialysis in a patient with renal failure in combination with diabetes, obesity, hyperlipidemia or a combination thereof, wherein said method comprises introducing a peritoneal dialysis solution comprising N-acetylglucosamine as an osmotic agent into the peritoneal cavity of the patient.

5. The method of claim 4, wherein the dialysis reduces or prevents hyperglycemia, hyperinsulinemia, hypertension or a combination thereof.

6. A method of reducing complications associated with peritoneal dialysis in a patient with diabetes, obesity, hyperlipidemia or a combination thereof, said method comprising introducing a peritoneal dialysis solution comprising N-acetylglucosamine as an osmotic agent into the peritoneal cavity of the patient.

7. The method of claim 6 wherein the NAG is present at a concentration of about 0.5 to 5.0% (w/v).

8. The method of claim 7 wherein the peritoneal dialysis solution further comprises at least one additional osmotic agent selected from the group consisting of glucose, iduronic acid, glucuronic acid, and a combination thereof.

9. The method of claim 6 wherein the complications asociated with peritoneal dialysis consist of:
(a) morphological and functional deterioration of the peritoneal membrane;
(b) peritonitis;
(c) adverse metabolic consequences and realted cardiovascular disease;
(d) protein malnutrition;
(e) hyperglycemia;
(f) hyperinsulinemia;
(g) hypertension; and combinations thereof.
